(19) United States
(12) Patent Application Publication
(10) Pub. No.: US 2023/0165435 A1
(43) Pub. Date: Jun. 1, 2023

(54) OPERATION UNIT AND ENDOSCOPE

(71) Applicant: FUJIFILM Corporation, Tokyo (JP)

(72) Inventors: Kunihiko TANAKA, Kanagawa (JP); Shintaro Hattori, Kanagawa (JP); Yutaka Senda, Kanagawa (JP)

(73) Assignee: FUJIFILM Corporation, Tokyo (JP)

(21) Appl. No.: 18/057,151

(22) Filed: Nov. 18, 2022

(30) Foreign Application Priority Data
Nov. 30, 2021 (JP) 2021-194571

(51) Int. Cl. A61B 1/00; A61B 1/05

(52) U.S. Cl. CPC A61B 1/00042; A61B 1/05; A61B 1/00096; A61B 1/00066

(57) ABSTRACT

There are provided an operation unit that allows a practitioner to easily ascertain a vertical direction of an observation image output from an image pickup unit and displayed on a monitor and an endoscope including the operation unit.

An operation unit is connected to a proximal end side of an insertion unit of an endoscope, and the insertion unit is provided with an optical system and an image pickup unit picking up an image of light passing through the optical system. The operation unit includes: a grip part that extends in a direction of an insertion axis of the insertion unit; a first flat surface portion that is formed on an outer surface of the grip part at a position on a top side in a vertical direction, extends in the direction of the insertion axis, and is perpendicular to the vertical direction in a case where a direction which indicates a top and a bottom of an image formed from image pickup signals output from the image pickup unit, among directions perpendicular to the direction of the insertion axis, is defined as the vertical direction; and a second flat surface portion that is formed on the outer surface of the grip part at a position on a bottom side in the vertical direction, extends in the direction of the insertion axis, and is perpendicular to the vertical direction.

### CROSS-REFERENCE TO RELATED APPLICATION

The present application claims priority under 35 U.S.C § 119(a) to Japanese Patent Application No. 2021-194571 filed on Nov. 30, 2021, which is hereby expressly incorporated by reference, in its entirety, into the present application.

### BACKGROUND OF THE INVENTION

### 1. Field of the Invention

The present invention relates to an operation unit that is connected to a proximal end side of an insertion unit of an endoscope and an endoscope comprising the operation unit.

### 2. Description of the Related Art

A rigid endoscope is known as an endoscope used for endoscopic surgery or the like (see WO2018/021583A). Further, an oblique-viewing endoscope of which a diagonal front side with respect to an insertion axis of an insertion unit corresponds to a visual field direction (an observation direction, an image pickup direction) is known as this rigid endoscope. The oblique-viewing endoscope comprises an insertion unit that is to be inserted into a patient's body, an operation unit that is connected to a proximal end side of the insertion unit, an optical system that is provided in a distal end portion of the insertion unit, and an image pickup unit that picks up an image of light transmitted through the optical system. An observation image picked up by the image pickup unit is output to a monitor through a cable. Accordingly, a practitioner can observe the inside of the patient's body through a monitor. Such oblique-viewing endoscopes in which a practitioner can operate the operation unit to change a visual field direction are disclosed in JP2021-510103A, U.S. Pat. No. 5,621,830A, and JP2018-32014A.

Each of operation units of the oblique-viewing endoscopes disclosed in JP2021-510103A and U.S. Pat. No. 5,621,830A includes a cylindrical grip part (handle) that is gripped by a practitioner, and a rotational operation member (a rotary swivel, an actuator) that is provided on a distal end side of the handle and that is rotatable in a direction around an insertion axis of the insertion unit. With regard to the oblique-viewing endoscopes disclosed in JP2021-510103A and U.S. Pat. No. 5,621,830A, a practitioner can rotationally operate the grip part to rotate the insertion unit in the direction around the insertion axis and to change the visual field direction of the oblique-viewing endoscope.

An operation unit of the oblique-viewing endoscope disclosed in JP2018-32014A includes a handle and a rotary wheel that is provided on a distal end side of the handle. In a case where a practitioner rotationally operates the handle in the oblique-viewing endoscope disclosed in JP2018-32014A, an optical system provided in a distal end portion of the insertion unit (endoscope shaft part) is rotated. Meanwhile, the rotary wheel prevents an image pickup unit, which is provided in the insertion unit, from rotating following the rotation of the optical system while the handle is rotationally operated. Accordingly, the rotation of a vertical direction indicating a top and a bottom of an observation image picked up by the oblique-viewing endoscope is prevented in a screen of the monitor.

### SUMMARY OF THE INVENTION

A practitioner (including an assistant, the same applies hereinafter) wishes to always ascertain the vertical direction of an observation image displayed on the monitor, and to perform a procedure while maintaining a state where the vertical direction is aligned with a vertical direction of the monitor (horizontality). However, a practitioner cannot ascertain the vertical direction of an observation image displayed on the monitor in the oblique-viewing endoscopes disclosed in JP2021-510103A and U.S. Pat. No. 5,621,830A.

Further, in the oblique-viewing endoscopes disclosed in JP2021-510103A and U.S. Pat. No. 5,621,830A, the insertion unit and the image pickup unit are integrally rotated with an operation for rotating the rotational operation member performed by a practitioner. For this reason, the vertical direction of an observation image displayed on the monitor also cannot be maintained constant in the oblique-viewing endoscopes disclosed in JP2021-510103A and U.S. Pat. No. 5,621,830A.

On the other hand, since the rotary wheel is provided in the oblique-viewing endoscope disclosed in JP2018-32014A, the vertical direction of an observation image displayed on the monitor can be maintained constant even though an operation for rotating the handle is performed. However, since a practitioner cannot ascertain the vertical direction of an observation image displayed on the monitor in the case of the operation unit disclosed in JP2018-32014A as in the cases of the operation units disclosed in JP2021-510103A and U.S. Pat. No. 5,621,830A, it is difficult to align the vertical direction of the observation image with the vertical direction of the monitor.

The present invention has been made in consideration of such circumstances, and an object of the present invention is to provide an operation unit that allows a practitioner to easily ascertain a vertical direction of an observation image output from an image pickup unit and displayed on a monitor and an endoscope including the operation unit.

An operation unit according to an aspect of the present invention is an operation unit that is connected to a proximal end side of an insertion unit of an endoscope, and the insertion unit is provided with an optical system and an image pickup unit picking up an image of light passing through the optical system. The operation unit comprises: a grip part that extends in a direction of an insertion axis of the insertion unit; a first flat surface portion that is formed on an outer surface of the grip part at a position on a top side in a vertical direction, extends in the direction of the insertion axis, and is perpendicular to the vertical direction in a case where a direction which indicates a top and a bottom of an image formed from image pickup signals output from the image pickup unit, among directions perpendicular to the direction of the insertion axis, is defined as the vertical direction; and a second flat surface portion that is formed on the outer surface of the grip part at a position on a bottom side in the vertical direction, extends in the direction of the insertion axis, and is perpendicular to the vertical direction. The image mentioned here is a monitor image that is output to a monitor (display unit) from the image pickup unit and that is displayed on the monitor.

According to this operation unit, a practitioner can easily ascertain the vertical direction of the image output from the image pickup unit.

According to another aspect of the present invention, in the operation unit, the grip part includes a first curved surface portion that connects a side edge portion of the first flat surface portion positioned on one side in a perpendicular direction to a side edge portion of the second flat surface portion positioned on the one side in the perpendicular direction and that bulges on the one side in the perpendicular direction in a case where a direction perpendicular to both the direction of the insertion axis and the vertical direction is defined as the perpendicular direction, and a second curved surface portion that connects a side edge portion of the first flat surface portion positioned on the other side in the perpendicular direction to a side edge portion of the second flat surface portion positioned on the other side in the perpendicular direction and that bulges on the other side in the perpendicular direction. Accordingly, a practitioner can easily ascertain the first flat surface portion and the second flat surface portion formed on the outer surface of the grip part, and it is possible to improve stability in a case where the practitioner grips the grip part.

According to another aspect of the present invention, in the operation unit, a proximal end portion of the grip part is formed in a shape of a dome. Since the proximal end portion is in contact with a palm of a practitioner's hand in a case where the practitioner grips the grip part, it is possible to improve stability in a case where the practitioner grips the grip part.

According to another aspect of the present invention, in the operation unit, the first flat surface portion is formed over a proximal end portion of the grip part from a distal end portion of the grip part, the second flat surface portion is formed over a position on a front side of the proximal end portion of the grip part from the distal end portion of the grip part, and a part of the proximal end portion of the grip part is a bulging portion that bulges on the bottom side of the second flat surface portion in the vertical direction. Accordingly, since a middle finger, a ring finger, or the like of a hand reaches the bulging portion in a case where a practitioner grips the grip part, it is possible to improve stability in a case where the practitioner grips the grip part.

According to another aspect of the present invention, the operation unit further comprises an inclined flat surface portion that is connected between a proximal end of the second flat surface portion and the bulging portion and that is inclined toward the bottom side in the vertical direction the further it extends toward a proximal end side from the proximal end of the second flat surface portion. Accordingly, since a middle finger, a ring finger, or the like of a hand reaches the inclined flat surface portion in a case where a practitioner grips the grip part, it is possible to improve stability in a case where the practitioner grips the grip part.

According to another aspect of the present invention, the operation unit further comprises a cable insertion portion which protrudes at a position offset to the bottom side in the vertical direction from a proximal apex of a proximal end portion of the grip part and into which a cable to be connected to the image pickup unit is inserted. As viewed in a perpendicular direction perpendicular to both the direction of the insertion axis and the vertical direction, the cable insertion portion protrudes in a direction that corresponds to a proximal end side of the proximal end portion of the grip part and is inclined toward the bottom side in the vertical direction with respect to the direction of the insertion axis. Accordingly, a pull-out allowance for an external cable at the proximal end portion can be ensured, and contact between the cable and a patient and a practitioner can be prevented.

According to another aspect of the present invention, the operation unit further comprises: an inner sheath-fixing part that is provided in the grip part not to be rotatable relative to the grip part in a direction around the insertion axis and that fixes a proximal end side of an inner sheath in a case where the insertion unit includes an outer pipe held on a distal end side of the grip part to be relatively rotatable in the direction around the insertion axis, a protection sheath inserted into the outer pipe and rotating in the direction around the insertion axis integrally with the outer pipe, and an inner sheath inserted into the protection sheath and rotatable relative to the outer pipe and to the protection sheath in the direction around the insertion axis, the optical system is provided on a distal end side of the protection sheath, and the image pickup unit is provided on a distal end side of the inner sheath; and an annular rotational operation member that is fixed to a proximal end side of the outer pipe and that rotates the outer pipe in the direction around the insertion axis.

According to another aspect of the present invention, in the operation unit, the grip part is made of a rubber material or a resin material. Accordingly, the grip part is less likely to slip in a hand.

An endoscope according to another aspect of the present invention comprises: an insertion unit that is provided with an optical system and an image pickup unit picking up an image of light passing through the optical system; and the above-mentioned operation unit that is connected to a proximal end side of the insertion unit.

According to another aspect of the present invention, in the endoscope, the insertion unit includes an outer pipe held on a distal end side of the grip part to be relatively rotatable in a direction around the insertion axis, a protection sheath inserted into the outer pipe and rotating in the direction around the insertion axis integrally with the outer pipe, an inner sheath inserted into the protection sheath and rotatable relative to the outer pipe and to the protection sheath in the direction around the insertion axis, and an inner sheath-fixing part that is provided in the grip part not to be rotatable relative to the grip part in the direction around the insertion axis and that fixes a proximal end side of the inner sheath, the optical system is provided on a distal end side of the protection sheath, and the image pickup unit is provided on a distal end side of the inner sheath.

According to another aspect of the present invention, the endoscope further comprises an annular rotational operation member that is fixed to a proximal end side of the outer pipe and that rotates the outer pipe in the direction around the insertion axis.

According to another aspect of the present invention, in the endoscope, the optical system includes a refractive optical element refracting light incident in a direction which is inclined with respect to the insertion axis, in a direction parallel to the insertion axis.

According to the present invention, a practitioner can easily ascertain a vertical direction of an image that is output from an image pickup unit and displayed on a monitor.

### BRIEF DESCRIPTION OF THE DRAWINGS

FIG. 1 is a diagram showing a configuration of an endoscope system that comprises an oblique-viewing endoscope.

FIG. 2 is an enlarged cross-sectional view of a distal end portion of an insertion unit.

FIG. 3 is a cross-sectional view of a main portion of a grip part and a knob.

FIG. 4 is a cross-sectional view of a protection sheath and a case.

FIG. 5 is an enlarged cross-sectional view of the case and a tubular portion.

FIG. 6 is a diagram illustrating a relationship between a vertical direction that indicates a top and a bottom of an image pickup system and a vertical direction that indicates a top and a bottom of an observation image displayed on a monitor.

FIG. 7 is a side view of an operation unit.

FIG. 8 is a top view of the operation unit.

FIG. 9 is a bottom view of the operation unit.

### DESCRIPTION OF THE PREFERRED EMBODIMENTS

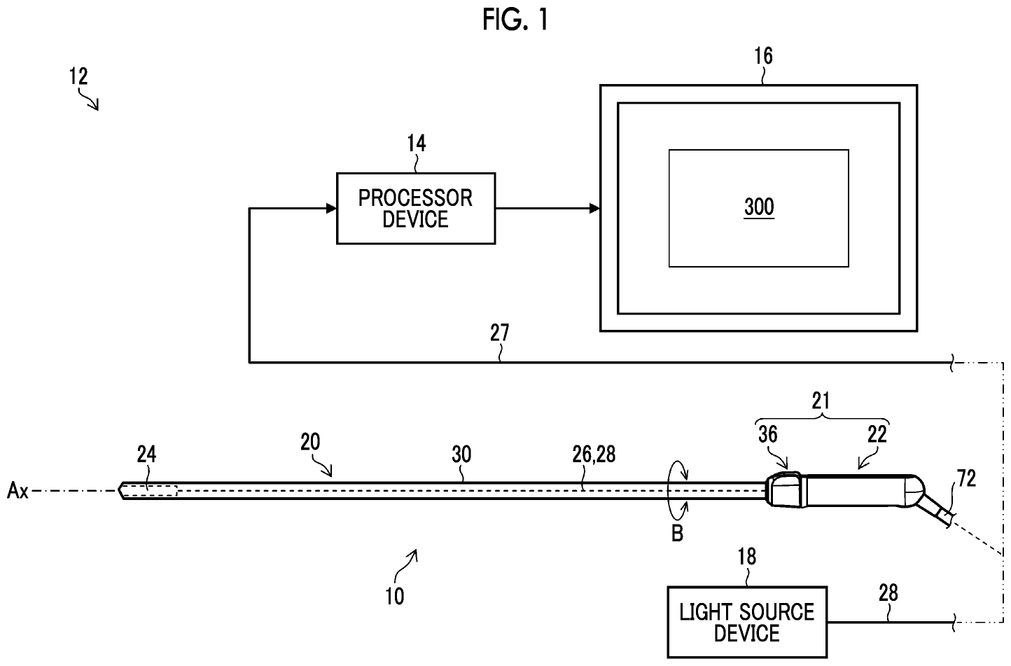

FIG. 1 is a diagram showing the configuration of an endoscope system 12 that comprises an oblique-viewing endoscope 10. As shown in FIG. 1, the endoscope system 12 comprises the oblique-viewing endoscope 10, a processor device 14, a monitor 16, and a light source device 18. The oblique-viewing endoscope 10 is an example of an endoscope of the present invention.

The oblique-viewing endoscope 10 is a so-called rigid endoscope, and comprises an insertion unit 20 and an operation unit 21. The insertion unit 20 is formed in a tubular shape (the shape of a pipe), and is to be inserted into a patient's body. The insertion unit 20 has a distal end, a proximal end, and an insertion axis Ax (also referred to as a longitudinal axis), and an outer peripheral wall of the insertion unit 20 is formed by an outer pipe 30 (also referred to as a sheath pipe) to be described later. A camera unit 24 to be described later is provided in a distal end portion of the insertion unit 20. Further, a first signal cable 26 and a light guide 28 are inserted into the insertion unit 20.

The first signal cable 26 connects the camera unit 24 to be described later to the processor device 14 together with a second signal cable 27 to be described later. A distal end portion of the first signal cable 26 is connected to the camera unit 24, and a proximal end portion of the first signal cable 26 is connected to the second signal cable 27 in the operation unit 21. A distal end portion (light emitting end surface) of the light guide 28 is provided on a distal end surface of the insertion unit 20, and a proximal end portion (light incident end surface) thereof is connected to the light source device 18. In this embodiment, a multi-core cable in which a plurality of strands (signal lines) are bundled, a shield conductor is provided around the strands, and the strands and the shield conductor are housed in a tubular sheath is exemplified as each of the first signal cable 26 and the second signal cable 27 that are an example of a cable of the present invention.

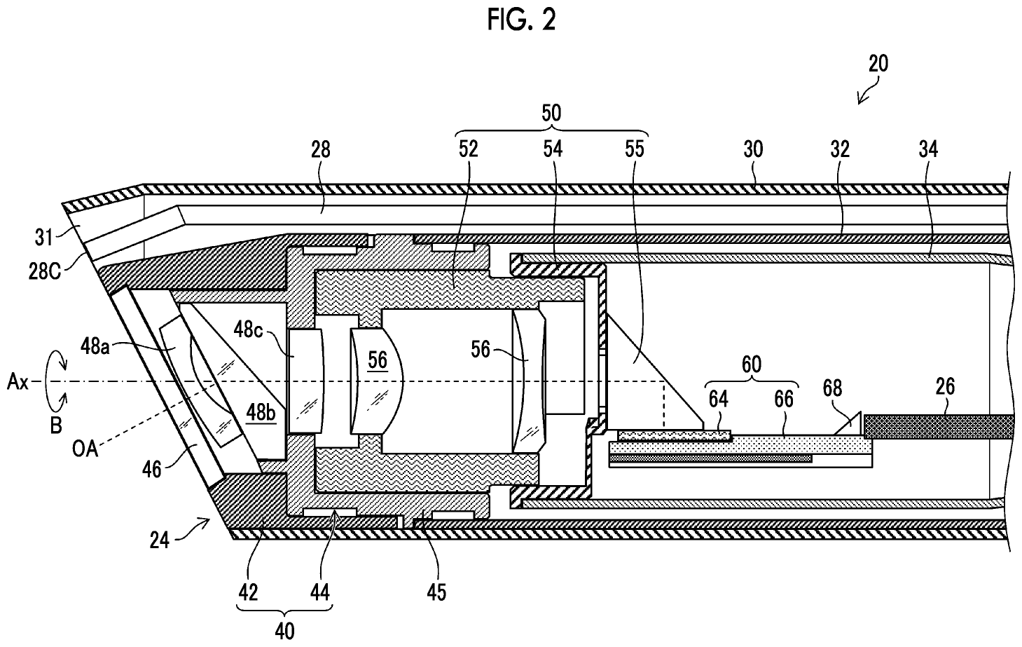

The light guide 28 has a light emitting end 28C (see FIG. 2) on a distal end side thereof, and the light emitting end 28C is disposed on a distal end side of the outer pipe 30. Further, the light guide 28 has a light incident end (not shown) on a proximal end side thereof, and the light incident end is connected to the light source device 18. For example, one optical cable in which a plurality of optical fibers are bundled is employed as the light guide 28, and has flexibility.

The operation unit 21 is connected to the proximal end side of the insertion unit 20. The operation unit 21 is gripped by a practitioner during an operation of the oblique-viewing endoscope 10, and receives a rotating operation for rotating a visual field direction of the oblique-viewing endoscope 10 (see an optical axis OA shown in FIG. 2) in a direction B around the insertion axis Ax, that is, a circumferential direction of the insertion unit 20 and of the operation unit 21, from the practitioner. The operation unit 21 includes a tubular grip part 22 that is gripped by the practitioner and a tubular (annular) knob 36 that receives a rotating operation for rotating the visual field direction. The knob 36 is an example of a rotational operation member of the present invention.

The grip part 22 has a size that fits the practitioner's hand, and is made of a rubber material or a resin material that withstands autoclave sterilization. Examples of such a rubber material include silicone rubber, fluororubber, and the like. Further, examples of the resin material include polyphenylsulfone (PPSU), poly ether ether ketone (PEEK), and the like. Accordingly, the grip part 22 is less likely to slip in the hand, that is, is less likely to rotate in the direction B around the axis as compared to a case where the grip part 22 is made of a metal material.

The outer pipe 30 is held at a distal end portion of the grip part 22 to be rotatable in the direction B around the axis. Further, an external cable 72 is connected to a proximal end portion of the grip part 22. The second signal cable 27 and the light guide 28 already described are inserted into the external cable 72.

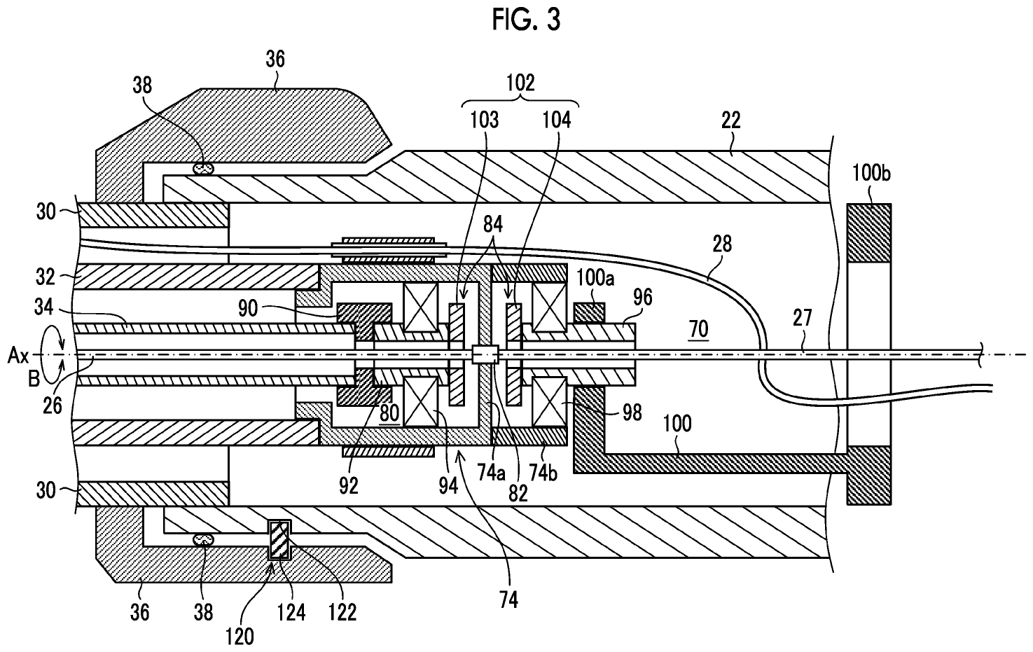

Further, as described in detail later, the grip part 22 includes an airtight space and a non-airtight space therein, and the proximal end portion of the first signal cable 26 and a distal end portion of the second signal cable 27 are connected to each other at a boundary between both the spaces (see FIG. 3). A proximal end portion of the second signal cable 27 is connected to the processor device 14. Accordingly, the camera unit 24 and the processor device 14 are electrically connected to each other via the first signal cable 26 and the second signal cable 27.

The knob 36 is fixed to a proximal end side of the outer pipe 30, so that the knob 36 is provided between the insertion unit 20 and the grip part 22. The knob 36 is a member that is used to change the visual field direction of the oblique-viewing endoscope 10 by rotating the outer pipe 30 relative to the grip part 22 in the direction B around the axis.

The processor device 14 generates an observation image 300 (video) of the inside of the patient's body on the basis of image pickup signals, which are input from the camera unit 24 through the first signal cable 26 and the second signal cable 27, and causes the monitor 16 to display this observation image 300. The observation image 300 corresponds to an example of an image of the present invention.

The light source device 18 supplies illumination light to the light guide 28. Accordingly, illumination light is emitted from the light emitting end 28C (see FIG. 2) of the light guide 28 that is provided on the distal end surface of the insertion unit 20.

FIG. 2 is an enlarged cross-sectional view of the distal end portion of the insertion unit 20. As shown in FIG. 2, the insertion unit 20 comprises the outer pipe 30, a protection sheath 32, and an inner sheath 34 that are formed substantially in the shape of a tube parallel to the insertion axis Ax. The outer pipe 30 forms the outer peripheral wall of the insertion unit 20 as already described. An opening of a distal end portion of the outer pipe 30 is inclined from a posture perpendicular to the insertion axis Ax. Further, as described in detail later, a proximal end portion of the outer pipe 30 is held by the distal end portion of the grip part 22 to be rotatable in the direction B around the axis. Furthermore, the knob 36 is externally fitted and fixed to the proximal end portion of the outer pipe 30.

The protection sheath 32 is inserted into and disposed in the outer pipe 30. A distal end optical system 40 of the camera unit 24 to be described later is provided in a distal end portion of the protection sheath 32. Further, as described in detail later, a proximal end portion of the protection sheath 32 is connected to a case 74 (see FIG. 3) provided in the grip part 22. Furthermore, a space 31 in which the light guide 28 is to be disposed is formed between an inner peripheral surface of the outer pipe 30 and an outer peripheral surface of the protection sheath 32.

The inner sheath 34 is inserted into and disposed in the protection sheath 32. The first signal cable 26 is inserted into the inner sheath 34. A proximal end optical system 50 and an image pickup unit 60 of the camera unit 24 to be described later are provided in a distal end portion of the inner sheath 34. Further, as described in detail later, a proximal end portion of the inner sheath 34 is connected to a connection member 90 (see FIG. 3) provided in the operation unit 21.

The camera unit 24 comprises the distal end optical system 40, the proximal end optical system 50, and the image pickup unit 60. Reference character OA shown in FIG. 2 denotes the optical axis of the optical system of the camera unit 24.

The distal end optical system 40 is an example of an optical system of the present invention, and is provided in the distal end portion of the protection sheath 32. The distal end optical system 40 is an oblique-viewing optical system that refracts light, which is incident in a direction inclined with respect to the insertion axis Ax, in a direction parallel to the insertion axis Ax and that guides the light to the proximal end optical system 50. The distal end optical system 40 includes a distal end portion body 42 and a distal end lens barrel 44 that is provided in the distal end portion body 42.

The distal end portion body 42 forms the distal end portion of the insertion unit 20 (protection sheath 32) and is a cap that covers the distal end lens barrel 44. Further, the distal end portion body 42 is formed substantially in the shape of a tube parallel to the insertion axis Ax. Furthermore, a cover glass 46, which is in an inclined posture corresponding to an inclination angle of an objective lens 48a provided in the distal end lens barrel 44 to be described later, is provided at a distal end-side opening portion of the distal end portion body 42.

Further, the distal end portion body 42 is fixed to the inner peripheral surface of the outer pipe 30. Accordingly, in a case where the outer pipe 30 is rotated in the direction B around the axis, the distal end optical system 40 and the protection sheath 32 are integrally rotated in the direction B around the axis together with the outer pipe 30.

The objective lens 48a, a prism 48b, and a lens 48c are housed in the distal end lens barrel 44. The objective lens 48a is inclined from a posture perpendicular to the insertion axis Ax and faces the cover glass 46. The objective lens 48a emits light, which is incident through the cover glass 46, toward the prism 48b. The prism 48b is an example of a refractive optical element of the present invention, and refracts light incident from the objective lens 48a, that is, light incident in a direction inclined with respect to the insertion axis Ax, in a direction parallel (including substantially parallel) to the insertion axis Ax and then emits the light toward the lens 48c. Accordingly, the visual field direction of the oblique-viewing endoscope 10 is inclined with respect to the insertion axis Ax. The lens 48c is in a posture perpendicular to the insertion axis Ax, and emits light incident from the prism 48b toward lenses 56 that are provided in a proximal end lens barrel 52 of the proximal end optical system 50 to be described later.

The configuration of an optical system provided in the distal end lens barrel 44 is not particularly limited as long as light incident in a direction inclined with respect to the insertion axis Ax can be guided into the proximal end lens barrel 52.

A tubular portion 45, which extends toward a proximal end side of the distal end lens barrel 44, is formed at the distal end lens barrel 44. This tubular portion 45 is externally fitted to be rotatable relative to a distal end portion of the proximal end lens barrel 52 to be described later in the direction B around the axis. Accordingly, the proximal end lens barrel 52 is fitted to be rotatable relative to the distal end lens barrel 44 in the direction around the axis.

The proximal end optical system 50 is provided in the distal end portion of the inner sheath 34, and guides light, which is incident from the distal end lens barrel 44, to the image pickup unit 60. The proximal end optical system 50 includes the proximal end lens barrel 52, a holder 54, and a prism 55.

The proximal end lens barrel 52 is fixed to the distal end portion of the inner sheath 34 via the holder 54. Further, the distal end portion of the proximal end lens barrel 52 is fitted to be rotatable relative to a proximal end-side opening portion of the tubular portion 45 in the direction B around the axis as already described. Accordingly, one of the distal end lens barrel 44 and the proximal end lens barrel 52 is rotatable relative to the other thereof in the direction B around the axis. As a result, the inner sheath 34 is rotatable relative to the protection sheath 32 in the direction B around the axis.

A plurality of lenses 56 having an optical axis OA parallel to the insertion axis Ax are provided in the proximal end lens barrel 52. Each lens 56 emits light, which is incident from the distal end lens barrel 44, toward the prism 55.

The holder 54 is formed substantially in the shape of a tube parallel to the insertion axis Ax, and is fixed to the distal end portion of the inner sheath 34. Further, the holder 54 is connected and fixed (externally fitted and fixed) to a proximal end portion of the proximal end lens barrel 52. Accordingly, since the inner sheath 34 and the proximal end lens barrel 52 are connected to each other by the holder 54, the inner sheath 34, the proximal end lens barrel 52, and the holder 54 are integrally rotatable relative to the protection sheath 32 in the direction B around the axis.

The prism 55 is held at a proximal end-side opening portion of the holder 54, and the image pickup unit 60 to be described later is held via the prism 55. For this reason, the image pickup unit 60 is rotatable relative to the protection sheath 32 in the direction B around the axis integrally with the inner sheath 34 and the proximal end lens barrel 52 via the holder 54 and the prism 55.

The prism 55 refracts light, which is incident through the proximal end lens barrel 52, by an angle of 90°. A mirror may be used instead of the prism 55.

The image pickup unit 60 picks up the image of the light (observation image 300) that passes through the distal end lens barrel 44 and the proximal end lens barrel 52 and is reflected by the prism 55. The image pickup unit 60 comprises an image pickup element 64 and a circuit board 66.

The image pickup element 64 is connected (fixed) to the prism 55 in a state where the image pickup element 64 is mounted on the circuit board 66, and is mounted on the holder 54 via the prism 55. Further, the image pickup element 64 picks up the image of the light, which is refracted by the prism 55, and outputs image pickup signals. A charge coupled device (CCD) image sensor or a complementary metal oxide semiconductor (CMOS) image sensor is used as the image pickup element 64.

The image pickup element 64 is mounted on the holder 54 via the prism 55 in this embodiment, but the image pickup element 64 may be directly mounted on the proximal end-side opening portion of the holder 54. In this case, since the image pickup element 64 is held in a posture perpendicular to the insertion axis Ax (optical axis OA) by the holder 54, the image pickup element 64 has a light-receiving surface perpendicular to the optical axis OA.

The circuit board 66 controls the drive of the image pickup element 64. Further, the distal end portion of the first signal cable 26 is connected to the circuit board 66 via a connector 68. Furthermore, the circuit board 66 outputs the image pickup signals of the image pickup element 64 to the first signal cable 26 via the connector 68.

FIG. 3 is a cross-sectional view of a main portion of the grip part 22 and the knob 36. As shown in FIG. 3, the grip part 22 is formed in the shape of a tube parallel to the insertion axis Ax.

The knob 36 fixed to the proximal end side of the outer pipe 30 is provided on the distal end side of the grip part 22. For example, the knob 36 is rotatably provided on an outer peripheral surface of the distal end portion of the grip part 22 via a seal ring 38. Accordingly, in a case where the knob 36 is operated to rotate in the direction B around the axis, the outer pipe 30 is rotated relative to the grip part 22 in the direction B around the axis, and the protection sheath 32 and the distal end optical system 40 are rotated in the same direction via the outer pipe 30. Therefore, the visual field direction (observation direction) of the oblique-viewing endoscope 10 can be changed. A rotational operation range of the knob 36 is regulated in a predetermined range (for example, 340°) by a rotation stopper 120.

The proximal end portions of the protection sheath 32 and the inner sheath 34 are inserted into the grip part 22 from a distal end-side opening portion of the grip part 22. Further, the external cable 72 already described is connected to the proximal end portion of the grip part 22. Furthermore, a light guide-insertion space 70 is formed in the grip part 22. Moreover, the case 74 is provided in the grip part 22. The case 74 is disposed on a distal end side of the light guide-insertion space 70.

The case 74 is formed substantially in the shape of a tube parallel to the insertion axis Ax to have a diameter smaller than the inner diameter of the grip part 22 and is housed in the grip part 22. The case 74 is held in the internal space of the grip part 22 by the protection sheath 32, a connecting beam 100 to be described later, and the like. The proximal end portion of the protection sheath 32 is connected to a distal end portion of the case 74. Accordingly, in a case where the outer pipe 30 is rotated relative to the grip part 22 in the direction B around the axis, this rotational force is transmitted to the distal end optical system 40, the protection sheath 32, and the case 74. As a result, the case 74 is rotated in the same direction as the outer pipe 30.

A proximal end side of the inner sheath 34 and a proximal end side of the first signal cable 26 are disposed in the case 74. Further, a partition wall 74a perpendicular to the insertion axis Ax is provided in the case 74, for example, in a proximal end-side opening portion of the case 74. The partition wall 74a closes the proximal end-side opening portion of the case 74.

Furthermore, a tubular portion 74b parallel to the insertion axis Ax is provided on a proximal end side of the case 74. The tubular portion 74b is formed to have the same diameter as the case 74, but may be formed to have a diameter different from the diameter of the case 74. Further, the tubular portion 74b may be formed integrally with the case 74. In this situation, a proximal end portion of the case 74 functions as a tubular portion 74b. A part of a connecting unit 84 to be described later is disposed in the case 74, and the distal end portion of the second signal cable 27 other than a part of the connecting unit 84 is disposed in the tubular portion 74b.

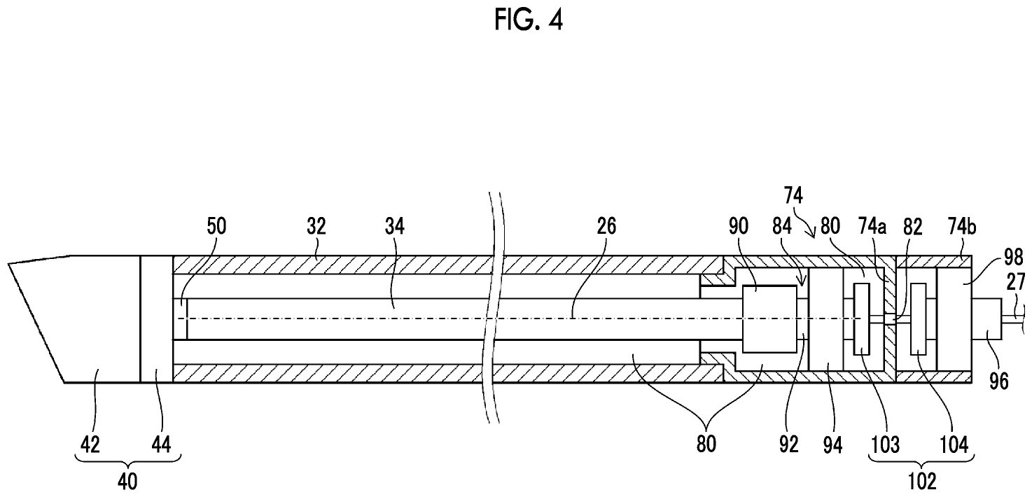

FIG. 4 is a cross-sectional view of the protection sheath 32 and the case 74. A sealed space 80 (airtight space) is formed in the protection sheath 32 and the case 74 as shown in FIG. 4, and the inner sheath 34, the image pickup unit 60, the first signal cable 26, and the like are disposed in the sealed space 80. A distal end side of the sealed space 80 is defined by the distal end optical system 40. Further, a proximal end side of the sealed space 80 is defined by the partition wall 74a. Accordingly, the moisture-proof property of the camera unit 24 is improved, so that fogging and breakage are prevented.

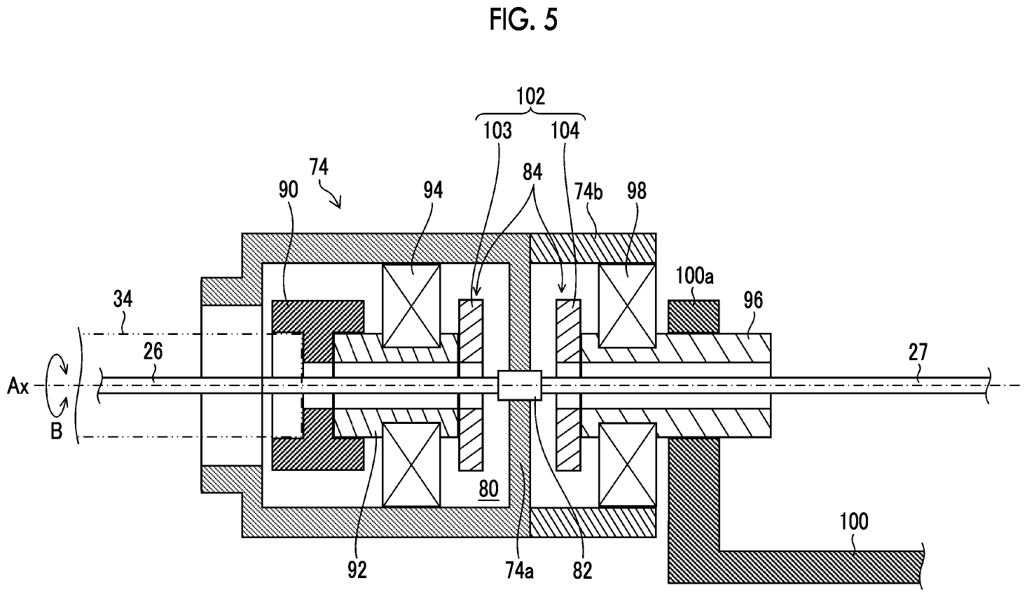

FIG. 5 is an enlarged cross-sectional view of the case 74 and the tubular portion 74b. As shown in FIGS. 3 to 5, the partition wall 74a already described, an airtight connector 82, and a connecting unit 84 are provided in the case 74 and the tubular portion 74b.

The airtight connector 82 is provided to pass through the inside and outside of the sealed space 80 and to be rotatable relative to the partition wall 74a in the direction B around the axis. The airtight connector 82 electrically connects the proximal end side of the first signal cable 26 provided in the case 74 (in the sealed space 80) to the distal end side of the second signal cable 27 provided in the tubular portion 74b (outside the sealed space 80). Accordingly, the first signal cable 26 and the second signal cable 27 are inserted into and disposed in the grip part 22. In a case where the first signal cable 26 and the second signal cable 27 are torsionally deformable in the direction B around the axis, for example, in a case where each of the first signal cable 26 and the second signal cable 27 is formed of a plurality of separated strands, the airtight connector 82 may be fixed to the partition wall 74a.

The connecting unit 84 is provided in the case 74 and the tubular portion 74b to be rotatable relative to the case 74 and to the tubular portion 74b in the direction B around the axis. The first signal cable 26 and the second signal cable 27 are inserted into the connecting unit 84. The connecting unit 84 magnetically connects the proximal end side of the inner sheath 34 provided in the case 74 (in the sealed space 80) to a connecting beam 100 to be described later provided outside the sealed space 80 with the partition wall 74a interposed therebetween.

The connecting unit 84 comprises a connection member 90, a bearing receiving member 92, and a bearing 94. Further, the connecting unit 84 comprises a bearing receiving member 96, a bearing 98, a connecting beam 100, and a magnet coupling 102 in addition to the above-mentioned members.

The connection member 90 and the bearing receiving member 92 are provided in the case 74 (in the sealed space 80), and are formed substantially in the shape of a tube parallel to the insertion axis Ax. Further, the first signal cable 26 is inserted into the connection member 90 and the bearing receiving member 92.

The connection member 90 connects the proximal end side of the inner sheath 34 to a distal end side of the bearing receiving member 92 in the case 74 (in the sealed space 80). Accordingly, the bearing receiving member 92 is connected to the inner sheath 34 via the connection member 90.

The distal end side of the bearing receiving member 92 is connected to the connection member 90 as described above, and a proximal end side thereof is fixed to a first magnet 103 of the magnet coupling 102. Further, the bearing 94, which is to be inscribed in the case 74, is fixed to an outer peripheral surface of the bearing receiving member 92. Accordingly, the bearing receiving member 92 and the first magnet 103 are held in the case 74 to be rotatable relative to the case 74 in the direction B around the axis. Various publicly known radial bearings, such as a ball bearing and a roller bearing, are used as the bearing 94.

The bearing receiving member 96 is provided in the tubular portion 74b (outside the sealed space 80). The bearing receiving member 96 is formed substantially in the shape of a tube parallel to the insertion axis Ax, and the second signal cable 27 is inserted into the bearing receiving member 96.

A distal end side of the bearing receiving member 96 is fixed to a second magnet 104 of the magnet coupling 102 in the tubular portion 74b, and a proximal end side thereof is connected to the connecting beam 100. Further, the bearing 98, which is to be inscribed in the tubular portion 74b, is fixed to an outer peripheral surface of the bearing receiving member 96. Accordingly, the bearing receiving member 96 and the second magnet 104 are held in the tubular portion 74b to be rotatable relative to the tubular portion 74b in the direction B around the axis. Various publicly known radial bearings are also used as the bearing 98 as in the case of the bearing 94.

Returning to FIG. 3, the connecting beam 100 is formed in the shape of a beam that extends in the direction of the insertion axis Ax in the light guide-insertion space 70. The connecting beam 100 includes a ring portion 100a provided on a distal end side thereof and a ring portion 100b provided on a proximal end side thereof. The ring portion 100a is externally fitted to a proximal end side of the bearing receiving member 96, and the ring portion 100b is fixed not to be relatively rotatable in the grip part 22 in the direction B around the axis. As a result, the inner sheath 34 (image pickup unit 60) is fixed via the connecting beam 100, the bearing receiving member 96, the magnet coupling 102 to be described later, and the bearing receiving member 92 not to be rotatable relative to the grip part 22. For this reason, the connecting beam 100 and the like are an example of an inner sheath-fixing part of the present invention.

The magnet coupling 102 includes the first magnet 103 provided in the case 74 (in the sealed space 80) and the second magnet 104 provided in the tubular portion 74b (outside the sealed space 80) with the partition wall 74a interposed therebetween. The magnet coupling 102 is a magnetic connecting member that magnetically connects the bearing receiving member 92 (inner sheath 34) to the bearing receiving member 96 (connecting beam 100). The first magnet 103 and the second magnet 104 have the shape of a disk parallel to the partition wall 74a (perpendicular to the insertion axis Ax). An insertion hole (not shown) into which the first signal cable 26 is to be inserted is formed at a central portion of the first magnet 103, and an insertion hole (not shown) into which the second signal cable 27 is to be inserted is formed at a central portion of the second magnet 104.

Since the inner sheath 34 and the connecting beam 100 are magnetically connected to each other via the magnet coupling 102, torque (stop torque) can be transmitted to the inner sheath 34 from the grip part 22. Accordingly, in a case where a practitioner rotationally operates the outer pipe 30 using the knob 36, the rotation (co-rotation) of the protection sheath 32 and the inner sheath 34 (the proximal end optical system 50 and the image pickup unit 60) in the direction B around the axis is prevented, that is, the posture of the inner sheath 34 in the direction B around the axis is maintained by the magnet coupling 102.

Next, the external shape of the operation unit 21 will be specifically described. As already described, a practitioner wishes to ascertain a vertical direction indicating the top and the bottom of the observation image 300, which is output from the image pickup unit 60 via the processor device 14 (hereinafter, simply paraphrased as “output from the image pickup unit 60”) and is displayed on the monitor 16, and to perform a procedure while maintaining a state where the vertical direction of the observation image 300 on the monitor 16 is aligned with a certain direction, for example, a vertical direction indicating the top and the bottom of the monitor 16. Accordingly, the grip part 22 of this embodiment has an external shape that allows a practitioner to easily ascertain the vertical direction of the observation image 300 displayed on the monitor 16. Further, the grip part 22 also has an external shape that improves usability, such as the ease of gripping the grip part 22 by a practitioner and a sense of stability in a case where a practitioner grips the grip part 22.

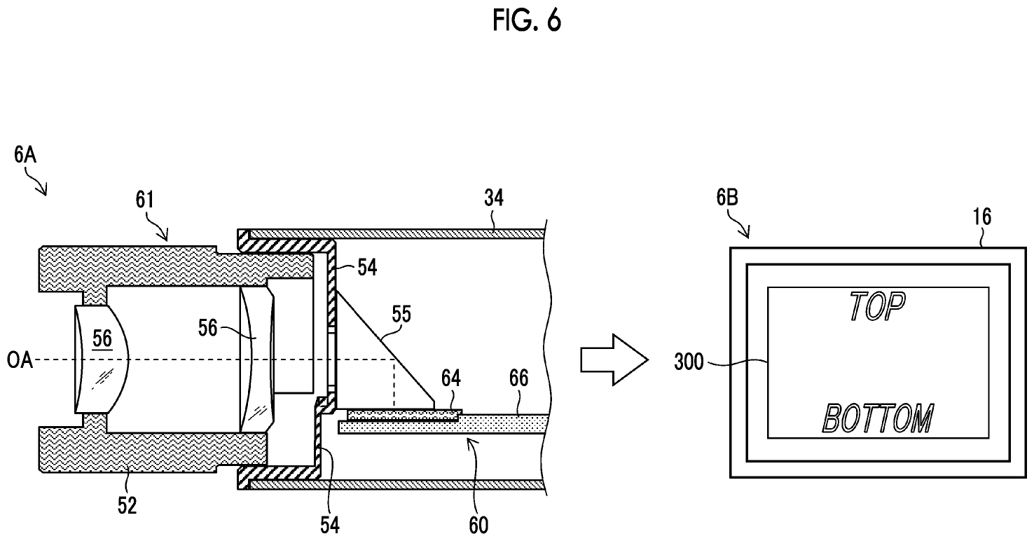

FIG. 6 is a diagram illustrating a relationship between a vertical direction that indicates the top and the bottom of an image pickup system 61 and a vertical direction that indicates the top and the bottom of the observation image 300 displayed on the monitor 16. Reference numeral 6A of FIG. 6 denotes a cross-sectional view of the image pickup system 61 (also referred to as an image pickup unit) that includes the proximal end optical system 50 (see FIG. 2) and the image pickup unit 60 provided in the distal end portion of the inner sheath 34. Reference numeral 6B of FIG. 6 denotes a front view of the observation image 300 displayed on the monitor 16.

As shown in FIG. 6, the vertical direction of the image pickup system 61 is determined on the basis of a direction corresponding to a top side (TOP) of the observation image 300 displayed on the monitor 16 and a direction corresponding to a bottom side (BOTTOM) of the observation image 300 on the monitor 16. The vertical direction of the image pickup system 61 can be arbitrarily set in relation to the monitor 16. In this embodiment, the light-receiving surface of the image pickup element 64 is disposed in a direction along the insertion axis Ax, and a vertical direction of the light-receiving surface of the image pickup element 64 is a left-right direction in 6A of FIG. 6 (a direction along the insertion axis Ax). Further, in this specification, the vertical direction of the image pickup system 61 means the vertical direction of the observation image 300 that is output from the image pickup unit 60 and displayed on the monitor 16 (hereinafter, simply paraphrased as “the vertical direction of the observation image 300”). Furthermore, in the vertical direction of the image pickup system 61, a direction corresponding to the top side (TOP) of the observation image 300 is defined as a top side direction of the vertical direction, and a direction corresponding to the bottom side (BOTTOM) of the observation image 300 is defined as a bottom side direction of the vertical direction.

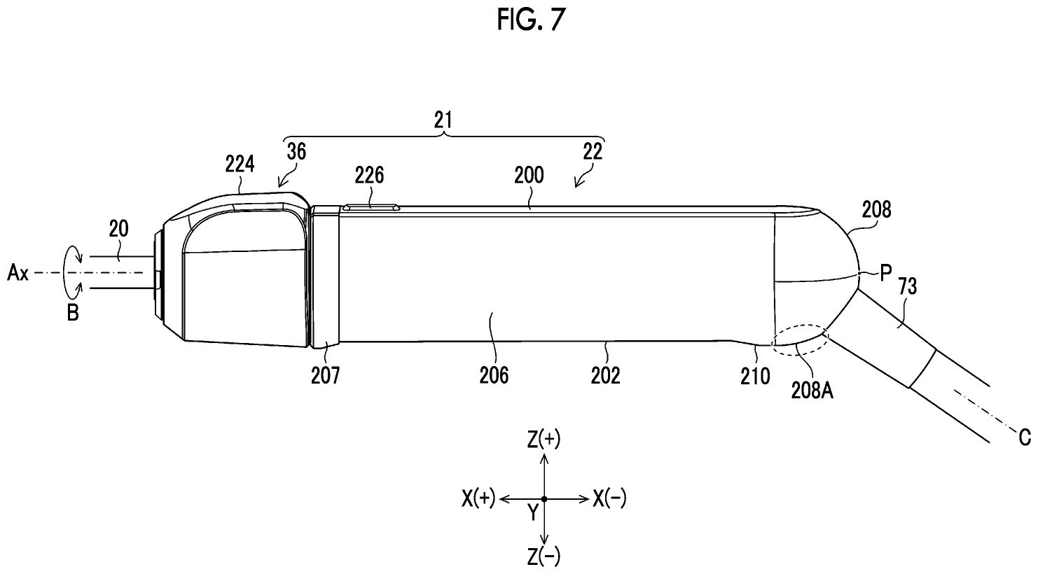

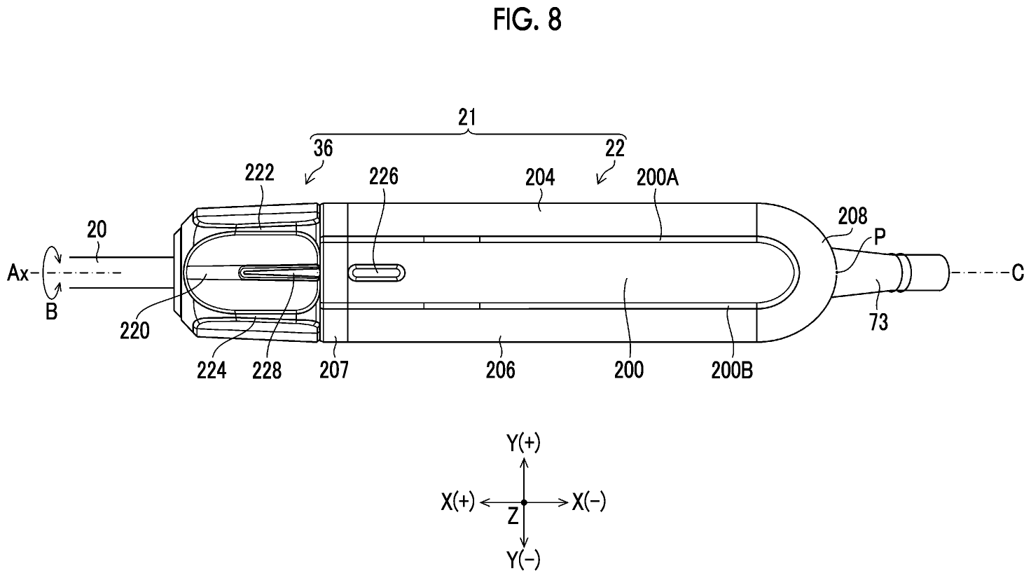

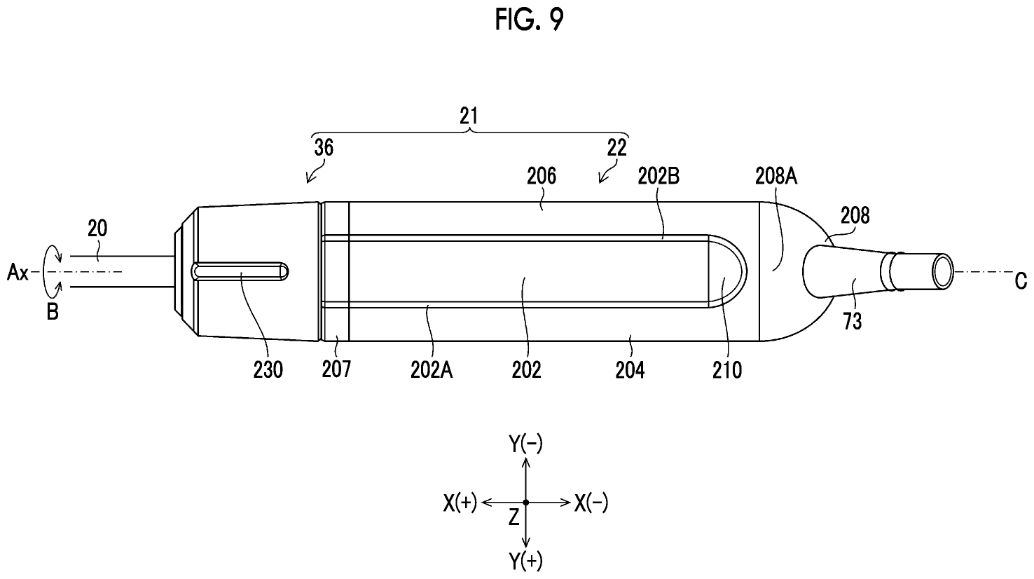

FIG. 7 is a side view of the operation unit 21. FIG. 8 is a top view of the operation unit 21. FIG. 9 is a bottom view of the operation unit 21. In FIGS. 7 to 9, among X, Y, and Z directions orthogonal to each other, a direction parallel to the insertion axis Ax is defined as an X direction, the vertical direction of the observation image 300 already described (the vertical direction of the image pickup system 61) is defined as a Z direction, and a direction perpendicular to both the X direction and the Z direction is defined as a Y direction. Further, in the X direction, a direction corresponding to a distal end side of the operation unit 21 is defined as an X(+) direction, and a direction corresponding to a proximal end side of the operation unit 21 is defined as an X(−) direction. Furthermore, in the Z direction, the top side direction of the vertical direction of the image pickup system 61 already described is defined as a Z(+) direction, and the bottom side direction of the vertical direction of the image pickup system 61 is defined as a Z(−) direction. In addition, any one side in the Y direction corresponds to a Y(+) direction, and the other side in the Y direction corresponds to a Y(−) direction.

In this specification, “the palm of the hand” means substantially the entire front portion of the hand excluding fingers. Further, “palm” means the central region of the palm of the hand (a concave portion between the hypothenar and the thenar).

As shown in FIGS. 7 to 9, the grip part 22 extends in the X direction, and is formed substantially in the shape of a tube of which a distal end portion 207 positioned on an X(+) side is open and a proximal end portion 208 positioned on an X(−) side is closed (see FIG. 3). A first flat surface portion 200, a second flat surface portion 202, a first curved surface portion 204, and a second curved surface portion 206 are formed on the outer surface of the grip part 22.

The first flat surface portion 200 is formed on the outer surface of the grip part 22 at a position on a Z(+) side that is a position on the top side in the vertical direction of the observation image 300 (the vertical direction of the image pickup system 61). The first flat surface portion 200 is a flat surface that extends in the X direction and that is perpendicular to the Z direction, and is formed over the proximal end portion 208 from the distal end portion 207 of the grip part 22. In a case where a practitioner grips the grip part 22, a base portion of a thumb of a practitioner's hand is in contact with the first flat surface portion 200. The first flat surface portion 200 may be substantially parallel to the X direction or may be substantially perpendicular to the Z direction.

Further, a first index 226 is formed at a distal end portion of the first flat surface portion 200. In a case where the first flat surface portion 200 is viewed from the Z(+) side, the first index 226 is positioned in the middle of the first flat surface portion 200 in the Y direction. As long as a practitioner can perceive the first index 226 with the tactile sensation of the practitioner's thumb or the like, the shape of the first index 226 is not particularly limited, and the first index 226 is formed in, for example, a convex shape.

The second flat surface portion 202 is formed on the outer surface of the grip part 22 at a position on a side opposite to the first flat surface portion 200 with the insertion axis Ax interposed between the first flat surface portion 200 and the second flat surface portion 202, that is, at a position on a Z(−) side that is a position on the bottom side in the vertical direction of the observation image 300 (the vertical direction of the image pickup system 61). The second flat surface portion 202 is a flat surface that extends in the X direction and that is perpendicular to the Z direction like the first flat surface portion 200, but is formed over a position on the front side of the proximal end portion 208 from the distal end portion 207. In a case where a practitioner grips the grip part 22, the fingers of the practitioner's hand other than the thumb reach the second flat surface portion 202. The second flat surface portion 202 may also be substantially parallel to the X direction or may also be substantially perpendicular to the Z direction.

The first curved surface portion 204 is a curved surface that connects a side edge portion 200A of the first flat surface portion 200 positioned on a Y(+) side to a side edge portion 202A of the second flat surface portion 202 positioned on the Y(+) side. The first curved surface portion 204 bulges on the Y(+) side. In a case where a practitioner grips the grip part 22 with, for example, a right hand, particularly, the palm of the right hand is in contact with the first curved surface portion 204.

The second curved surface portion 206 is a curved surface that connects a side edge portion 200B of the first flat surface portion 200 positioned on a Y(−) side to a side edge portion 202B of the second flat surface portion 202 positioned on the Y(−) side. The second curved surface portion 206 bulges on the Y(−) side. In a case where a practitioner grips the grip part 22 with, for example, the right hand, the tips of the fingers of the right hand other than the thumb are in contact with the second curved surface portion 206.

The proximal end portion 208 of the grip part 22 is formed in the shape of a dome (referred to as the shape of a cannonball) bulging in the X(−) direction. In a case where a practitioner grips the grip part 22, the proximal end portion 208 is in contact with the palm of the practitioner's hand.

Further, since the second flat surface portion 202 is formed up to the front side of the proximal end portion 208 as already described, a part of the proximal end portion 208, that is, a part of the proximal end portion 208 positioned on the Z(−) side, forms a bulging portion 208A that bulges on the Z(−) side of the second flat surface portion 202. Furthermore, an inclined flat surface portion 210 is connected between the bulging portion 208A and the second flat surface portion 202. The inclined flat surface portion 210 is an inclined surface that is inclined toward the Z(−) side the further it extends toward the X(−) side from a proximal end of the second flat surface portion 202. In a case where a practitioner grips the grip part 22, a middle finger or a ring finger (or a little finger) of the practitioner's hand can reach the bulging portion 208A and the inclined flat surface portion 210.

A cable insertion portion 73 having substantially the shape of a pipe protrudes from the proximal end portion 208 at a position that is offset to the Z(−) side from a proximal apex P of the proximal end portion 208 closest to the proximal end side. In a case where the cable insertion portion 73 is viewed in the Y direction, the cable insertion portion 73 is inclined in a diagonally downward direction C that is inclined to the X(−) side from the proximal end portion 208 and to the Z(−) side with respect to the X(−) direction. The external cable 72 is connected to the cable insertion portion 73. Accordingly, the second signal cable 27 and the light guide 28, which are present in the external cable 72, are inserted into the grip part 22 through the cable insertion portion 73. As a result, the second signal cable 27 is connected to the first signal cable 26 in the grip part 22, and is electrically connected to the image pickup unit 60 via the first signal cable 26. Further, the light emitting end 28C of the light guide 28 is disposed on the distal end side of the outer pipe 30 through the light guide-insertion space 70 and the space 31.

In a case where a practitioner grips the grip part 22, the cable insertion portion 73 is in contact with the palm of the practitioner's hand. Accordingly, the external cable 72 is led out from a base portion of the little finger of the practitioner's hand gripping the grip part 22.

A finger placing portion 220, finger rest portions 222 and 224, a second index 228, and a third index 230 are formed on an outer surface of the knob 36.

A ball of the practitioner's thumb gripping the grip part 22 is placed on the finger placing portion 220. The shape of the finger placing portion 220 is the shape of a concavely curved surface to correspond to the shape of the ball of the thumb.

The finger rest portions 222 and 224 are formed on the outer surface of the knob 36 such that the finger placing portion 220 is interposed between the finger rest portions 222 and 224 in the direction B around the axis. The finger rest portions 222 and 224 are in contact with both side portions of the thumb placed on the finger placing portion 220. Accordingly, an operating force generated in a case where a practitioner moves the thumb in a left-right direction can be efficiently transmitted to the knob 36.

The second index 228 is provided on the finger placing portion 220. As long as a practitioner can perceive the second index 228 with the practitioner's thumb, the shape of the second index 228 is not particularly limited, and the second index 228 is formed in, for example, a convex shape. In a case where the rotational position of the knob 36 in the direction B around the axis is adjusted to the middle position (neutral position) of the rotation range of the knob 36, the second index 228 is aligned with the same line as the first index 226, which is formed on the first flat surface portion 200, in the X direction. Accordingly, the practitioner can easily ascertain the middle position of the knob 36 with the feeling of a finger. Further, in a case where the rotational position of the knob 36 is adjusted such that the second index 228 coincides with the first index 226, the visual field direction (an observation direction, an image pickup direction) of the oblique-viewing endoscope 10 includes a component corresponding to the Z(−) direction.

The third index 230 is formed on the outer surface of the knob 36 at a position on a side opposite to a position where the second index 228 is formed with the insertion axis Ax interposed between the second index 228 and the third index 230. In a case where the knob 36 is rotated relative to the grip part 22 from the middle position in the direction B around the axis by a large angle (for example, 120°), the third index 230 can be visually observed even though the second index 228 cannot be visually observed. For this reason, the rotational position of the knob 36 can be ascertained on the basis of the third index 230.

Next, an action of the oblique-viewing endoscope 10 having the above-mentioned configuration, particularly, the operation unit 21, will be described.

In a case where a practitioner uses the oblique-viewing endoscope 10 to observe the inside of a patient's body or to perform a procedure, the practitioner grips the grip part 22 with, for example, the right hand (or a left hand). In this case, the base portion of the thumb of the right hand is in contact with the first flat surface portion 200, the fingers of the right hand other than the thumb reach the second flat surface portion 202, the inclined flat surface portion 210, and the bulging portion 208A, the palm of the right hand is in contact with the first curved surface portion 204 and the proximal end portion 208, the tips of the fingers of the right hand other than the thumb are in contact with the second curved surface portion 206, and the cable insertion portion 73 is in contact with the palm of the right hand. Further, the external cable 72 is led out from the base portion of the little finger of the right hand.

In a case where the base portion of the thumb of the practitioner's right hand is placed on the first flat surface portion 200, it is easy to support the second flat surface portion 202 with the other fingers of the right hand. Accordingly, it is easy for the practitioner to grip the grip part 22. Further, in a case where the practitioner places the thumb on the first flat surface portion 200 of the grip part 22, the practitioner can place the ball of the thumb on the finger placing portion 220 in a natural state along the first flat surface portion 200. Furthermore, in a case where the palm of the right hand is turned up, the grip part 22 is prevented from rolling on the palm of the right hand since the second flat surface portion 202 is formed on the outer surface of the grip part 22. Moreover, since the proximal end portion 208 is in contact with the palm of the right hand, it is possible to improve stability in a case where the practitioner grips the grip part 22.

In addition, since the middle finger, the ring finger, or the like of the right hand reaches the inclined flat surface portion 210 and the bulging portion 208A in a case where a practitioner grips the grip part 22, it is possible to further improve stability in a case where the practitioner grips the grip part 22. Further, since the second flat surface portion 202 is formed up to a position on the front side of the proximal end portion 208 and the inclined flat surface portion 210 and the bulging portion 208A are provided, a pull-out allowance for the external cable 72 at the proximal end portion 208 can be ensured.

Furthermore, in a case where a practitioner grips the grip part 22, the external cable 72 can be led out in the diagonally downward direction C from the proximal end portion 208 by the cable insertion portion 73. In a case where the external cable 72 is led out to the Z(−) side from the proximal end portion 208, the external cable 72 is in contact with a patient. In a case where the external cable 72 is led out to the X(−) side from the proximal end portion 208, the external cable 72 is in contact with the practitioner. However, the occurrence of these problems can be avoided in this embodiment.

As described above, the grip part 22 of this embodiment has an external shape that improves usability, such as the ease of gripping the grip part 22 by a practitioner and a sense of stability in a case where a practitioner grips the grip part 22. As a result, since the rotation of the grip part 22 in the practitioner's hand is prevented, a change in the vertical direction of the observation image 300 is prevented.

After gripping the grip part 22, a practitioner inserts the insertion unit 20 into a patient's body and checks the observation image 300 output from the image pickup unit 60 on the monitor 16. Accordingly, the practitioner can observe the inside of the patient's body through the monitor 16. Further, in a case where a practitioner is to change the visual field direction of the oblique-viewing endoscope 10, the practitioner rotationally operates the knob 36 in the direction B around the axis via the thumb of the right hand placed on the finger placing portion 220. Accordingly, the outer pipe 30 and the protection sheath 32 (distal end optical system 40) are rotated integrally with the knob 36 in the same direction, so that the visual field direction of the oblique-viewing endoscope 10 can be directed to a desired direction.

Since the posture of the inner sheath 34 in the direction B around the axis is maintained by the magnet coupling 102 in this case, the rotation (co-rotation) of the protection sheath 32 and the inner sheath 34 (the proximal end optical system 50 and the image pickup unit 60) in the direction B around the axis is prevented. As a result, since the rotation of the observation image 300 to be observed on the monitor 16 is prevented even though the visual field direction is changed, the vertical direction of the observation image 300 is maintained. Further, in the operation unit 21 of this embodiment, it is possible to rotationally operate the knob 36 with the right hand gripping the grip part 22, that is, it is possible to grip the operation unit 21 and to rotationally operate the knob 36 with one hand. As a result, the operability of the oblique-viewing endoscope 10 is improved.

A practitioner ascertains the vertical direction of the observation image 300 on the basis of the first flat surface portion 200 and the second flat surface portion 202 formed on the outer surface of the grip part 22 while observing the inside of a patient's body using the observation image 300 displayed on the monitor 16. As already described, the first flat surface portion 200 is formed on the outer surface of the grip part 22 at a position on the top side in the vertical direction of the observation image 300, and the second flat surface portion 202 is formed on the outer surface of the grip part 22 at a position on the bottom side in the vertical direction of the observation image 300. For this reason, a practitioner can ascertain the vertical direction of the observation image 300 only with the feeling of the right hand (fingers) gripping the grip part 22 without looking away from the monitor 16. Accordingly, a practitioner can adjust the posture of the grip part 22 to align the vertical direction of the observation image 300 with a desired direction, such as the vertical direction of the monitor 16. As a result, a practitioner can maintain a state where the vertical direction of the observation image 300 is aligned with the vertical direction of the monitor 16 while performing the observation of the inside of a patient's body through the monitor 16, an operation for rotating the knob 36, another operation, or a procedure.

In this embodiment, as described above, the first flat surface portion 200 is formed on the outer surface of the grip part 22 at a position on the top side in the vertical direction of the observation image 300, and the second flat surface portion 202 is formed on the outer surface of the grip part 22 at a position on the bottom side in the vertical direction of the observation image 300. Accordingly, a practitioner can easily ascertain the vertical direction of the observation image 300.

Further, in a case where the shape of a surface other than the first flat surface portion 200 and the second flat surface portion 202 of the outer surface of the grip part 22 is set to the shape of a curved surface, that is, a shape other than the shape of a flat surface, a practitioner can easily ascertain the first flat surface portion 200 and the second flat surface portion 202, that is, the vertical direction of the observation image 300 only with the feeling of the fingers of the hand.

Other

The insertion unit 20 includes the outer pipe 30, the protection sheath 32, and the inner sheath 34 in the embodiment, but the configuration of the insertion unit 20 is not particularly limited as long as the visual field direction can be changed depending on an operation for rotating the knob 36.

In the embodiment, the side edge portion 200A and the side edge portion 202A are connected to each other by the first curved surface portion 204, and the side edge portion 200B and the side edge portion 202B are connected to each other by the second curved surface portion 206. As long as a practitioner can perceive the first flat surface portion 200 and the second flat surface portion 202 with the feeling of the hand, surfaces having arbitrary shapes may be provided instead of the first curved surface portion 204 and the second curved surface portion 206.

The case 74 is provided in the grip part 22 in the embodiment, but a component provided in the grip part 22 is not particularly limited.

The oblique-viewing endoscope 10 of which the visual field direction can be changed has been described as a rigid endoscope in the embodiment by way of example, but the present invention can also be applied to a rigid endoscope of which the visual field direction is fixed and to an operation unit thereof. In this case, the knob 36 is omitted from the operation unit 21. Further, the present invention is not limited to a rigid endoscope, and can also be applied to a flexible endoscope and to an operation unit thereof.

### EXPLANATION OF REFERENCES

10: oblique-viewing endoscope

12: endoscope system

14: processor device

16: monitor

18: light source device

20: insertion unit

21: operation unit

22: grip part

24: camera unit

26: first signal cable

27: second signal cable

28: light guide

28C: light emitting end

30: outer pipe

31: space

32: protection sheath

34: inner sheath

36: knob

38: seal ring

40: distal end optical system

42: distal end portion body

44: distal end lens barrel

45: tubular portion

46: cover glass

48a: objective lens

48b: prism

48c: lens

50: proximal end optical system

52: proximal end lens barrel

54: holder

55: prism

56: lens

60: image pickup unit

61: image pickup system

64: image pickup element

66: circuit board

68: connector

70: light guide-insertion space

72: external cable

73: cable insertion portion

74: case

74a: partition wall

74b: tubular portion

80: sealed space

82: airtight connector

84: connecting unit

90: connection member

92: bearing receiving member

94: bearing

96: bearing receiving member

98: bearing

100: connecting beam

100a: ring portion

100b: ring portion

102: magnet coupling

103: first magnet

104: second magnet

120: rotation stopper

200: first flat surface portion

200A: side edge portion

200B: side edge portion

202: second flat surface portion

202A: side edge portion

202B: side edge portion

204: first curved surface portion

206: second curved surface portion

207: distal end portion

208: proximal end portion

208A: bulging portion

210: inclined flat surface portion

220: finger placing portion

222: finger rest portion

224: finger rest portion

226: first index

228: second index

230: third index

300: observation image

Ax: insertion axis

B: direction around axis

C: diagonally downward direction

OA: optical axis

P: proximal apex

What is claimed is:

1. An operation unit that is connected to a proximal end side of an insertion unit of an endoscope, the insertion unit being provided with an optical system and an image pickup unit picking up an image of light passing through the optical system, the operation unit comprising:
a grip part that extends in a direction of an insertion axis of the insertion unit;
a first flat surface portion that is formed on an outer surface of the grip part at a position on a top side in a vertical direction, extends in the direction of the insertion axis, and is perpendicular to the vertical direction in a case where a direction which indicates a top and a bottom of an image formed from image pickup signals output from the image pickup unit, among directions perpendicular to the direction of the insertion axis, is defined as the vertical direction; and
a second flat surface portion that is formed on the outer surface of the grip part at a position on a bottom side in the vertical direction, extends in the direction of the insertion axis, and is perpendicular to the vertical direction.

2. The operation unit according to claim 1,
wherein the grip part includes
a first curved surface portion that connects a side edge portion of the first flat surface portion positioned on one side in a perpendicular direction to a side edge portion of the second flat surface portion positioned on the one side in the perpendicular direction and that bulges on the one side in the perpendicular direction in a case where a direction perpendicular to both the direction of the insertion axis and the vertical direction is defined as the perpendicular direction, and
a second curved surface portion that connects a side edge portion of the first flat surface portion positioned on the other side in the perpendicular direction to a side edge portion of the second flat surface portion positioned on the other side in the perpendicular direction and that bulges on the other side in the perpendicular direction.

3. The operation unit according to claim 1,
wherein a proximal end portion of the grip part is formed in a shape of a dome.

4. The operation unit according to claim 1,
wherein the first flat surface portion is formed over a proximal end portion of the grip part from a distal end portion of the grip part,
the second flat surface portion is formed over a position on a front side of the proximal end portion of the grip part from the distal end portion of the grip part, and
a part of the proximal end portion of the grip part is a bulging portion that bulges on the bottom side of the second flat surface portion in the vertical direction.

5. The operation unit according to claim 4, further comprising:
an inclined flat surface portion that is connected between a proximal end of the second flat surface portion and the bulging portion and that is inclined toward the bottom side in the vertical direction the further it extends toward a proximal end side from the proximal end of the second flat surface portion.

6. The operation unit according to claim 1, further comprising:
a cable insertion portion which protrudes at a position offset to the bottom side in the vertical direction from a proximal apex of a proximal end portion of the grip part and into which a cable to be connected to the image pickup unit is inserted,
wherein as viewed in a perpendicular direction perpendicular to both the direction of the insertion axis and the vertical direction, the cable insertion portion protrudes in a direction that corresponds to a proximal end side of the proximal end portion of the grip part and is inclined toward the bottom side in the vertical direction with respect to the direction of the insertion axis.

7. The operation unit according to claim 1, further comprising:
an inner sheath-fixing part that is provided in the grip part not to be rotatable relative to the grip part in a direction around the insertion axis and that fixes a proximal end side of an inner sheath in a case where the insertion unit includes an outer pipe held on a distal end side of the grip part to be relatively rotatable in the direction around the insertion axis, a protection sheath inserted into the outer pipe and rotating in the direction around the insertion axis integrally with the outer pipe, and an inner sheath inserted into the protection sheath and rotatable relative to the outer pipe and to the protection sheath in the direction around the insertion axis, the optical system is provided on a distal end side of the protection sheath, and the image pickup unit is provided on a distal end side of the inner sheath; and
an annular rotational operation member that is fixed to a proximal end side of the outer pipe and that rotates the outer pipe in the direction around the insertion axis.

8. The operation unit according to claim 1,
wherein the grip part is made of a rubber material or a resin material.

9. An endoscope comprising:
an insertion unit that is provided with an optical system and an image pickup unit picking up an image of light passing through the optical system; and
the operation unit according to claim 1 that is connected to a proximal end side of the insertion unit.

10. The endoscope according to claim 9,
wherein the insertion unit includes
an outer pipe held on a distal end side of the grip part to be relatively rotatable in a direction around the insertion axis,
a protection sheath inserted into the outer pipe and rotating in the direction around the insertion axis integrally with the outer pipe,
an inner sheath inserted into the protection sheath and rotatable relative to the outer pipe and to the protection sheath in the direction around the insertion axis, and
an inner sheath-fixing part that is provided in the grip part not to be rotatable relative to the grip part in the direction around the insertion axis and that fixes a proximal end side of the inner sheath,
the optical system is provided on a distal end side of the protection sheath, and
the image pickup unit is provided on a distal end side of the inner sheath.

11. The endoscope according to claim 10, further comprising:
an annular rotational operation member that is fixed to a proximal end side of the outer pipe and that rotates the outer pipe in the direction around the insertion axis.

12. The endoscope according to claim 9,
wherein the optical system includes a refractive optical element refracting light incident in a direction which is inclined with respect to the insertion axis, in a direction parallel to the insertion axis.
